(19) United States
(12) Patent Application Publication
(10) Pub. No.: US 2007/0173822 A1
(43) Pub. Date: Jul. 26, 2007

(54) Use of a posterior dynamic stabilization system with an intradiscal device

(75) Inventors: Aurelien Bruneau, Memphis, TN (US); Thomas Carls, Memphis, TN (US); Eric C. Lange, Collierville, TN (US); Fred J. Molz IV, Birmingham, AL (US); Matthew M. Morrison, Cordova, TN (US); Jonathan Dewey, Memphis, TN (US); Kent M. Anderson, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/331,924

(22) Filed: Jan. 13, 2006

(51) Int. Cl. A61F 2/30

(52) U.S. Cl. 606/61

(57) ABSTRACT

A method of treating a spinal condition includes attaching an anterior spinal motion device in an anterior region of a motion segment associated with the pair of vertebrae and attaching a posterior motion preservation device in a second region of the motion segment. The anterior spinal motion device may include an elastic material or a motion preserving disc prosthesis having at least one articulating surface.

### BACKGROUND

The present application relates to the following applications, all of which are filed concurrently herewith, assigned to the same assignee, and are hereby incorporated by reference.

| | Attorney | |
|---|---|---|
| Title | Docket No. | Inventor(s) |
| Materials, Devices, and Methods for | P22578.00 | Hai H. Trieu |
| Treating Multiple Spinal Regions Including | 31132.376 | |
| The Posterior and Spinous Process Regions | | |
| Materials, Devices, and Methods for | P22615.00 | Hai H. Trieu |
| Treating Multiple Spinal Regions Including | 31132.377 | |
| The Anterior Region | | |
| Materials, Devices, and Methods for | P22656.00 | Hai H. Trieu |
| Treating Multiple Spinal Regions Including | 31132.378 | |
| The Interbody Region | | |
| Materials, Devices, and Methods for | P22681.00 | Hai H. Trieu |
| Treating Multiple Spinal Regions Including | 31132.379 | |
| Vertebral Body and Endplate Regions | | |

Disease, degradation, and trauma of the spine can lead to various conditions that require treatment to maintain, stabilize, or reconstruct the vertebral column. As the standard of care in spine treatment begins to move from arthrodesis to arthroplasty, preserving motion and limiting further degradation in a spinal joint or in a series of spinal joints becomes increasingly more complex.

To date, standard treatments of the vertebral column have not adequately addressed the need for multiple devices, systems, and procedures to treat joint degradation. Likewise, current techniques do not adequately address the impact that a single treatment or arthroplasty device may have on the adjacent bone, soft tissue, or joint behavior.

For example, stand-alone anterior spinal motion devices (or dynamic stabilization devices) do not fully stabilize the spine; they permit motion while resisting anterior-column load. For this reason, interbody motion devices are sometimes ineffective when there is any posterior muscular, ligamentus, or other instability. On the other hand, posterior dynamic stabilization devices do not substantially resist loads through the anterior column, nor can they provide anterior distraction. Thus, while both anterior and posterior dynamic devices permit motion, each is capable of providing something the other cannot.

### SUMMARY

The present disclosure describes the use of a posterior dynamic stabilization system with an intradiscal device. In one embodiment, a method of treating a spinal condition includes attaching an anterior spinal motion (dynamic stabilization) device in an anterior region of a motion segment associated with the pair of vertebrae and attaching a posterior motion preservation device in a second region of the motion segment.

In some embodiments, the anterior spinal motion device may include an elastic material or a motion preserving disc prosthesis having at least one articulating surface.

In another embodiment, a kit for introduction into a single surgical environment is disclosed. The kit includes an anterior spinal motion device for attachment to an anterior region of a motion segment associated with a pair of adjacent vertebrae and a posterior motion preservation device for attachment to a second region of the motion segment.

In some embodiments, the anterior spinal motion device may be adapted to accommodate an existence of the posterior motion preservation device in the same motion segment. In some embodiments, the posterior motion preservation device is adapted to accommodate an existence of the anterior spinal motion device in the same motion segment.

### BRIEF DESCRIPTION OF THE DRAWINGS

FIG. 1 is a sagittal view of a section of a vertebral column.

FIG. 2 is a superior view of a vertebral body depicted in FIG. 1.

FIG. 3 is a block diagram of a multiple region treatment system according to one or more embodiments of the present invention.

FIGS. 4-5 are sagittal views of a section of a vertebral column having multiple region treatments.

### DETAILED DESCRIPTION

The present disclosure relates generally to vertebral reconstructive devices, and more particularly, to systems and procedures for treating multiple spinal regions. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The use of both anterior and posterior devices can be complimentary, thereby broadening the scope of dynamic stabilization. The present invention, including the embodiments discussed below, relates to the combined use of anterior/interbody motion-preserving implants, such as disc and nucleus arthroplasty, with posterior dynamic stabilization devices, including both fixed (e.g., pedicle screw based) and fixed or non-fixed (e.g., interspinous process and cable) implants. The disclosed embodiments allow the use of anterior spinal motion devices despite degradation in the facets and/or spinous process. The disclosed embodiments also allow the use of posterior dynamic stabilization devices despite advanced disc degeneration.

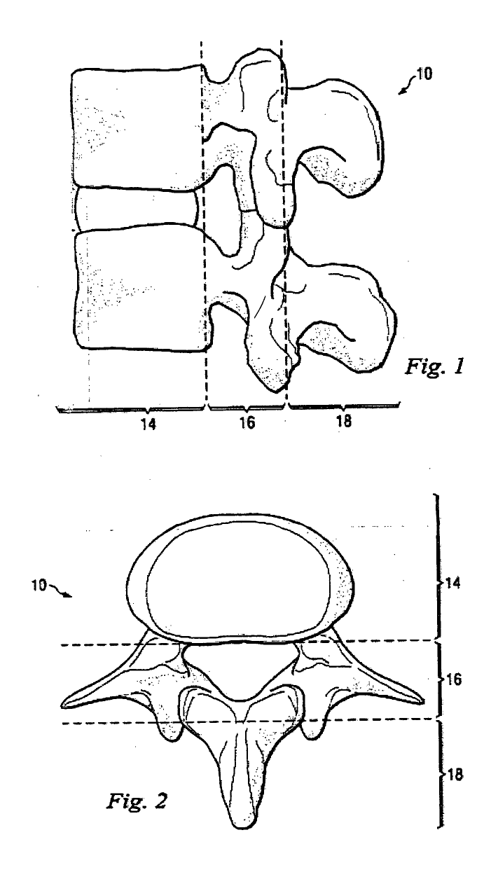

Referring first to FIGS. 1 and 2, the reference numeral 10 refers to a vertebral joint section or a “motion segment” of a vertebral column. As used herein, the term motion segment describes the overall relative movement between adjacent vertebrae. This includes movement at the disc space, at the facet joints, and movement allowed through various tissue, ligament and muscle compositions, as will be described in greater detail below.

The motion segment 10 may be considered as having several regions extending from anterior to posterior. These regions include an anterior column region 14, a posterior facet region 16, and a posterior spinous process region 18.

Disc degeneration may lead to disc collapse or loss of disc height, resulting in pain or neurodeficit. Similarly, degeneration of the facet joints may lead to pain or neurodeficit. When treating one degenerated region of the motion segment, the impact of the treatment on the surrounding regions should be considered. For example, inappropriate restoration of disc height to only a posterior portion of the interbody space may result in hyperkyphosis with loss of height in the anterior interbody area and placement of the anterior annulus in compression. Also, improvements to the anterior interbody area alone is difficult to achieve when instability such as spondylolisthesis or retrolisthesis exists. Likewise, in appropriate restoration of disc height to only an anterior portion of the interbody space may result in hyperlordosis with loss of posterior disc height and compression of the posterior annulus and facet joints.

Treatment, stabilization, and/or reconstruction of the vertebral motion segment 10 may be diagnosed and carried out in a systematic manner depending upon the conditions and material or devices available for treatment. To achieve an improved clinical outcome and a stable result, multiple regions of the vertebral column can be treated.

Anterior Column Region 14

The anterior column region 14 may require treatment due to disc collapse or loss of disc height due to degeneration, disease, or trauma. It is often desired to treat the anterior column region 14 by providing an anterior spinal motion device in the disc space. Disc space or intervertebral body devices and systems for treating region 14 include prosthetic motion preserving discs such as those offered by or developed by Medtronic, Inc. under brand names such as MAVERICK, BRYAN, PRESTIGE, or PRESTIGE LP. Single articulating surface motion preserving discs are disclosed more fully in U.S. Pat. Nos. 6,740,118; 6,113,637; or 6,540,785 which are incorporated by reference herein. Double articulating surface motion preserving discs are disclosed more fully in U.S. Pat. Nos. 5,674,296; 6,156,067; or 5,865,846 which are incorporated by reference herein. In some embodiments, prosthetic motion preserving discs may extend posteriorly from the interbody space and include features for providing posterior motion. These types of bridged devices are disclosed in U.S. Pub. Pat. App. Nos. 2005/0171610; 2005/0171609; 2005/0171608; 2005/0154467; 2005/0154466; 2005/0154465; 2005/0154464; 2005/0154461 which are incorporated by reference herein. In another embodiment, a spherical, ellipsoidal or similarly shaped disc replacement device may be installed in the interbody space. Such devices include the SATELLITE device offered by or developed by Medtronic, Inc. This type of device is described in detail, for example, in U.S. Pat. No. 6,478,822 which is incorporated by reference herein. In still another embodiment, a disc replacement device may be an elastically deformable device comprising a resilient or an elastomeric material and/or may comprise a mechanical spring component.

Alternatively, interbody motion preserving devices may include nucleus replacement implants that work in conjunction with all or portions of the natural annulus. Such nucleus replacement implants may include those offered by or developed by Medtronic, Inc under a brand name such as NAUTILUS or offered by or developed by Raymedica, Inc. of Minneapolis, Minn. under brand names such as PDN-SOLO® and PDN-SOLO XL™. These types of nucleus replacement implants are described in detail in, for example, U.S. Pat. Nos. 6,620,196 and 5,674,295, which are incorporated by reference herein. Injectable nucleus replacement material including a polymer based device such as DASCOR™ by Disc Dynamics of Eden Prairie, Minn. or a protein polymer device such as NuCore™ Injectable Nucleus by Spine Wave, Inc. of Shelton, Conn. may be alternatives for preserving interbody motion. Other acceptable alternative injectable or insertable disc augmentation biomaterials may be natural or synthetic and may include injectable and in situ curable polyurethane or an in situ curable poly vinyl alcohol compound. Injectable silicone or collagen may also be used to restore disc height and/or preserve joint motion. Injectable materials may be used alone or together with an inflatable container implanted within the interbody space.

The interbody devices may be loaded in compression or tension depending upon the patient's indication or the performance of other implanted devices or treatments. These interbody devices may provide a desired level of intervertebral disc space distraction the depending upon the patient's indication. For example, an interbody device may be sized or filled to balance posterior interspinous distraction provided by an interspinous device.

Posterior Facet Region 16

Posterior region devices for treating region 16 may extend along the posterior or posterolateral side of the vertebral column and may span one or more motion segments. Posterior devices may be used with intact anatomy or in situations in which one or more facet, the spinous process, or even the entire lamina have been resected. Examples of semi-rigid or flexible posterior devices include systems offered by or developed by Medtronic, Inc. under brand names such as FLEXTANT or AGILE or offered by or developed by Zimmer, Inc. of Warsaw, Ind. such as the Dynesys® Dynamic Stabilization System. These types of flexible devices are disclosed, for example, in U.S. Pat. Pub. Nos. 2005/0171540 and 2005/0131405, which are hereby incorporated by reference. These particular devices may replace or supplement natural facet joints and may attach to the posterior features of adjacent vertebrae using bone screws. Additional devices may include Archus Othopedics, Inc.'s TOTAL FACET ARTHROPLASTY SYSTEM (TFAS™) or similar devices performing facet functions

Alternatively, dampener devices such as those described in U.S. Pat. Nos. 5,375,823; 5,540,688; 5,480,401 or U.S. Pat. App. Pub. Nos. 2003/0055427 and 2004/0116927, each of which is incorporated by reference herein. Additionally, rod and screw systems that use flexible PEEK rods may be chosen. In another alternative, posterior devices may be made of flexible materials such as woven or braided textile based devices that connect with two or more vertebrae. These flexible materials may be formed of natural graft material or synthetic alternatives.

The posterior facet region devices may connect to two or more vertebral bodies or vertebral endplates through the use of any connection mechanism such as bone screws, staples, sutures, or adhesives. The systems and devices may be loaded in compression or tension depending upon the patient's indication or the performance of other implanted systems or treatments. For example, a flexible device attached to adjacent vertebrae with bone screws may be installed in tension to balance disc degeneration or subsidence of an interbody prosthesis.

The posterior facet region devices may be formed of less rigid or more flexible materials, may be formed of inelastic material, or of elastic material. The devices may be formed of composite material including one or more materials listed above.

Posterior Spinous Process Region 18

Spinous process devices for treating posterior region 18 may extend between adjacent spinous processes and/or extend around or through adjacent spinous processes. As one example, spinous process devices may include semi-rigid spacer systems having flexible interspinous process sections and flexible ligaments or tethers for attaching around or through spinous processes. Such devices may include the DIAM device offered by or developed by Medtronic, Inc. or the Wallis device offered by or developed by Abbott Laboratories of Abbott Park, Ill. Semi-rigid spacer devices are disclosed in greater detail in U.S. Pat. Nos. 6,626,944 and 6,761,720 which are incorporated by reference herein. Alternatively, semi-rigid spacer devices may have rigid interspinous process sections but incorporating flexible ligament or tethering devices that permit a limited amount of flexion-extension motion at the motion segment.

In other embodiments, spinous process devices may include artificial ligaments for connecting two or more spinous processes. In still other embodiments, interspinous process devices may be made of flexible materials such as tethers that connect with two or more vertebrae. Depending upon the device chosen, the spinous process devices may be installed through open surgical procedures, minimally invasive procedures, injection, or other methods known in the art. These systems and devices may be loaded in compression or tension depending upon the patient's indication or the performance of other implanted devices or treatments.

### EXAMPLES

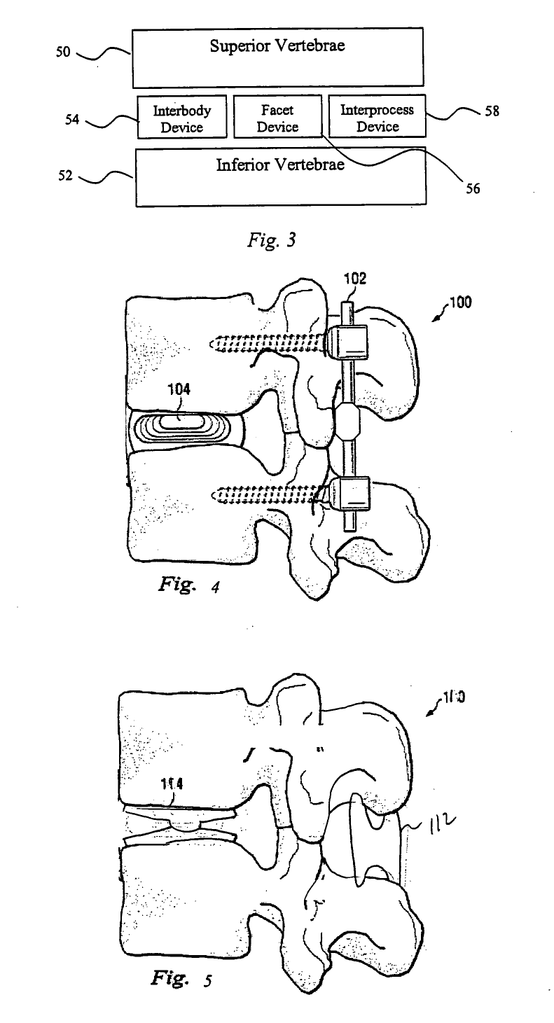

Referring now to FIG. 3, between two adjacent vertebrae 50, 52, various combinations of devices can be used to address needs of a patient. Such devices may include an interbody device 54 positioned in the anterior column region 14 (FIGS. 1 and 2), a facet device 56 in the posterior facet region 16, and/or an interprocess device 58 in the posterior spinous process region 18.

As shown in FIG. 4, in a more particular example, a multiple region system 100 may include a posterior motion device 102 such as described in presently incorporated U.S. Publication No. 2005/0131405. The system 100 may further include a nucleus replacement device 104 such as a NAUTILUS device offered by or developed by Medtronic, Inc. It is understood that the combination of treatment methods and devices described in FIG. 4 is merely exemplary and that other materials and systems may be chosen to achieve a desired result involving the posterior, intervertebral body, and anterior regions.

As shown in FIG. 5, in another example, a multiple region system 110 may include an interprocess device 112 such as the above-referenced DIAM device attached to adjacent spinous processes, and an interbody device 114 such as the above-referenced MAVERICK disc system attached to the adjacent vertebral bodies.

In some embodiments, adjustments and/or selections can be made to the various devices 54-58 (FIG. 3) in light of the affect of the combination of devices. For example, the interbody device 54 can be positioned in the disc space in a different location due to the added stabilization being provided by either of the other two devices 56, 58. In addition or in the alternative, one or more of the devices may be differently configured in light of the combined result. For example, an axis of articulation in the interbody device 54 can be moved towards the anterior due to the improved support from the other two devices 56, 58.

In several combinations, the two or more devices 54-58 may change in configuration throughout a surgical process. For example, the interbody device 54 may be implanted first, and be configured in a first position for engagement with the adjacent vertebrae 50, 52. One or more posterior devices 56, 58 may then be inserted through a separate access point. Once inserted, the previously inserted interbody device 54 may be configured in a second position to the effect of the one or more posterior devices. The devices 54, 56, and/or 58 can be configured or positioned into their corresponding region to facilitate this change of position during the surgical process.

Thus, materials, devices, and methods for treating multiple spinal regions are presently described. Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A method of treating a spinal condition comprising:
attaching an anterior spinal motion device in an anterior region of a motion segment associated with the pair of vertebrae; and
attaching a posterior motion preservation device in a second region of the motion segment.

2. The method of claim 1 wherein the anterior spinal motion device comprises an elastic material.

3. The method of claim 2 wherein the elastic material is an elastomer or a rubber.

4. The method of claim 1 wherein the anterior spinal motion device is attached to a more-anterior position of the anterior region, than an anterior spinal motion device that would be attached without an accompanying posterior motion preservation device.

5. The method of claim 1 wherein the anterior spinal motion device is configured to articulate in a more-anterior position of the anterior region, than an anterior spinal motion device that would be attached without an accompanying posterior motion preservation device.

6. The method of claim 1 wherein the anterior spinal motion device comprises a motion preserving disc prosthesis having at least one articulating surface.

7. The method of claim 1 wherein the anterior spinal motion device comprises a material injectable into a natural nucleus.

8. The method of claim 1 wherein the anterior spinal motion device comprises a nucleus replacement device.

9. The method of claim 8 wherein the nucleus replacement device is rigid.

10. The method of claim 8 wherein the nucleus replacement device is flexible.

11. The method of claim 1 wherein the posterior motion preservation device includes a facet-support device.

12. The method of claim 11 wherein the posterior motion preservation device includes a facet-replacement device.

13. The method of claim 1 wherein the posterior motion preservation device includes an interspinous process device.

14. The method of claim 13 wherein the interspinous process device includes a bumper device.

15. The method of claim 13 wherein the interspinous process device includes a facet-support device.

16. The method of claim 1 wherein at least one of either the anterior spinal motion device or the posterior motion preservation device includes a bone ingrowth surface adapted for securely engaging with one of the two vertebrae, and wherein the two devices are attached in the motion segment prior to secure engagement by the bone ingrowth surface.

17. The method of claim 1 wherein the anterior spinal motion device is constructed to be positioned according to a first configuration prior to the attachment of the posterior motion preservation device, and positioned in a second configuration after the attachment of the posterior motion preservation device.

18. A kit for introduction into a single surgical environment, the kit comprising:
an anterior spinal motion device for attachment to an anterior region of a motion segment associated with a pair of adjacent vertebrae; and
a posterior motion preservation device for attachment to a second region of the motion segment.

19. The kit of claim 18 wherein the anterior spinal motion device is adapted to accommodate an existence of the posterior motion preservation device in the same motion segment.

20. The kit of claim 18 wherein the posterior motion preservation device is adapted to accommodate an existence of the anterior spinal motion device in the same motion segment.
